(19) United States
(12) Patent Application Publication
(10) Pub. No.: US 2026/0024628 A1
(43) Pub. Date: Jan. 22, 2026

(54) APPARATUS AND METHOD FOR PREDICTING ABSORBANCE SPECTRUM

(71) Applicant: Electronics and Telecommunications Research Institute, Daejeon (KR)

(72) Inventors: Ji Yong HWANG, Daejeon (KR); Hyun Woo OH, Daejeon (KR)

(21) Appl. No.: 19/251,667

(22) Filed: Jun. 26, 2025

(30) Foreign Application Priority Data
Jul. 22, 2024 (KR) 10-2024-0096620

(51) Int. Cl. G16C 60/00; C09B 67/22

(52) U.S. Cl. CPC G16C 60/00; C09B 67/0033

(57) ABSTRACT

An apparatus for predicting an absorbance spectrum of a blended fabric including a first fabric and a second fabric includes a memory that stores at least one command, and a processor that performs the at least one command stored in the memory, wherein the processor predicts a first absorbance spectrum, which is an absorbance spectrum for a first dye used for dyeing of the first fabric, predicts a second absorbance spectrum, which is an absorbance spectrum for a second dye used for dyeing of the second fabric, corrects the first and second absorbance spectra based on a blending ratio of the blended fabric, and predicts a third absorbance spectrum, which is an absorbance spectrum of the blended fabric, based on the corrected first and second absorbance spectra.

### CROSS-REFERENCE TO RELATED APPLICATION

This application claims priority to and the benefit of Korean Patent Application No. 10-2024-0096620, filed on Jul. 22, 2024, the disclosure of which is incorporated herein by reference in its entirety.

### BACKGROUND

### 1. Field of the Invention

The present invention relates to an apparatus and method for predicting an absorbance spectrum, and more specifically, to an apparatus and method for predicting an absorbance spectrum that enable prediction of an absorbance spectrum of a blended fabric.

### 2. Discussion of Related Art

In order to reproduce a buyer's order color, a worker on site selects three to six monochromatic dyes used in a factory and calculates a dye mixing prescription through simulation. The dye mixing prescription is simulated based on previously input basic data. Based on several dye mixing prescriptions recommended through the simulation, a B/T test is conducted. Whether the color is reproduced in the B/T test is confirmed, and when the color is reproduced through the B/T test, a corresponding dye mixing prescription is issued to the site through a prescription. However, scale-up occurs since dyeing at the site is not at a laboratory level, and a color difference occurs due to various factors. When the color difference occurs, the color difference is corrected based on information on a deviation obtained from a comparison with buyer order data through computer color matching (CCM) colorimetry.

Conventionally, a dye prescription is calculated and corrected using various methods (simulation using a CCM colorimetry system, simulation using an AI model, or the like). One method for correcting a dye prescription is to correct the dye prescription so that an absorbance spectrum of a dye used for dyeing of a fabric matches an absorbance spectrum of a buyer order color. However, in the case of a blended fabric, since it is not easy to predict an absorbance spectrum, it is difficult to apply a dye prescription method based on the absorbance described above when a dye prescription for the blended fabric is corrected.

The background technology of the present invention is disclosed in Korean Patent No. 10-2035059 (Oct. 16, 2019).

### SUMMARY OF THE INVENTION

The present invention is directed to providing an apparatus and method for predicting an absorbance spectrum which enable prediction of an absorbance spectrum of a blended fabric based on an absorbance spectrum of each of fabrics constituting the blended fabric and a blending ratio of the blended fabric.

According to some embodiments of the present invention, an apparatus for predicting an absorbance spectrum is an apparatus for predicting an absorbance spectrum of a blended fabric including a first fabric and a second fabric, the apparatus including a memory configured to store at least one command, and a processor configured to perform the at least one command stored in the memory, wherein the processor predicts a first absorbance spectrum, which is an absorbance spectrum for a first dye used for dyeing of the first fabric, predicts a second absorbance spectrum, which is an absorbance spectrum for a second dye used for dyeing of the second fabric, corrects the first and second absorbance spectra based on a blending ratio of the blended fabric, and predicts a third absorbance spectrum, which is an absorbance spectrum of the blended fabric, based on the corrected first and second absorbance spectra.

In some embodiments of the present invention, the first dye may be a hydrophilic dye, and the second dye is a hydrophobic dye.

In some embodiments of the present invention, the first and second dyes may be a single dye or a mixed dye.

In some embodiments of the present invention, the processor may predict an absorbance spectrum of the first dye from information on a type and concentration of each of monochromatic dyes used for production of the first dye using a preset mathematical model.

In some embodiments of the present invention, the processor may correct the first absorbance spectrum by performing a process of multiplying an absorbance value by a first percentage, which is a percentage of the first fabric in the blended fabric, in an entire wavelength range of the first absorbance spectrum, and corrects the second absorbance spectrum by performing a process of multiplying the absorbance value by a second percentage, which is a percentage of the second fabric in the blended fabric, in an entire wavelength range of the second absorbance spectrum.

In some embodiments of the present invention, the processor may predict the third absorbance spectrum by performing a process of summing an absorbance value of the first dye and an absorbance value of the second dye in the entire wavelength range of the first and second absorbance spectra.

In some embodiments of the present invention, the processor may compare the third absorbance spectrum with a fourth absorbance spectrum, which is an absorbance spectrum for an electronic color value of a target color, and correct a dye prescription for the blended fabric based on a result of the comparison.

In some embodiments of the present invention, the processor may correct the dye prescription by performing at least one of an operation of replacing at least one of the first dye and the second dye with another dye, an operation of changing a concentration of at least one of the first dye and the second dye, and an operation of changing a mixing ratio of at least one of the first dye and the second dye.

According to some embodiments of the present invention, a method for predicting an absorbance spectrum is a method for predicting an absorbance spectrum of a blended fabric including a first fabric and a second fabric, which is performed in a computing device including a processor, the method including predicting a first absorbance spectrum, which is an absorbance spectrum of a first dye used for dyeing of the first fabric, predicting a second absorbance spectrum, which is an absorbance spectrum for a second dye used for dyeing of the second fabric, correcting the first and second absorbance spectra based on a blending ratio of the blended fabric, and predicting a third absorbance spectrum, which is the absorbance spectrum of the blended fabric, based on the corrected first and second absorbance spectra.

In some embodiments of the present invention, the first dye may be a hydrophilic dye, and the second dye is a hydrophobic dye.

In some embodiments of the present invention, the first and second dyes may be a single dye or a mixed dye.

In some embodiments of the present invention, the predicting of the first absorbance spectrum may include predicting an absorbance spectrum of the first dye from information on a type and concentration of each of monochromatic dyes used for production of the first dye using a preset mathematical model.

In some embodiments of the present invention, the correcting of the first and second absorbance spectra may include performing a process of multiplying an absorbance value by a first percentage, which is a percentage of the first fabric in the blended fabric, in an entire wavelength range of the first absorbance spectrum, and performing a process of multiplying the absorbance value by a second percentage, which is a percentage of the second fabric in the blended fabric, in an entire wavelength range of the second absorbance spectrum.

In some embodiments of the present invention, the predicting of the third absorbance spectrum may include performing a process of summing the absorbance of the first dye and the absorbance of the second dye in the entire wavelength range of the first and second absorbance spectra.

In some embodiments of the present invention, the method for predicting an absorbance spectrum may further include comparing the third absorbance spectrum with a fourth absorbance spectrum, which is an absorbance spectrum for an electronic color value of a target color, and correcting a dye prescription for the blended fabric based on a result of the comparison.

In some embodiments of the present invention, the correcting of the dye prescription may include performing at least one of an operation of replacing at least one of the first dye and the second dye with another dye, an operation of changing a concentration of at least one of the first dye and the second dye, and an operation of changing a mixing ratio of at least one of the first dye and the second dye.

According to some embodiments of the present invention, an apparatus for predicting an absorbance spectrum is an apparatus for predicting an absorbance spectrum of a blended fabric including a first fabric and a second fabric, the apparatus including a communication interface, and a processor connected to the communication interface, wherein the processor receives a first absorbance spectrum, which is an absorbance spectrum of a first dye used for dyeing of the first fabric and a second absorbance spectrum, which is an absorbance spectrum for a second dye used for dyeing of the second fabric via the communication interface, corrects the first and second absorbance spectra based on a blending ratio of the blended fabric, and predicts a third absorbance spectrum, which is an absorbance spectrum of the blended fabric, based on the corrected first and second absorbance spectra.

In some embodiments of the present invention, the first dye may be a hydrophilic dye, and the second dye may be a hydrophobic dye.

In some embodiments of the present invention, the processor may correct the first absorbance spectrum by multiplying the first absorbance spectrum by a first percentage, which is a percentage of the first fabric in the blended fabric, and correct the second absorbance spectrum by multiplying the second absorbance spectrum by a second percentage, which is a percentage of the second fabric in the blended fabric.

In some embodiments of the present invention, the processor may predict the third absorbance spectrum by summing the corrected first and second absorbance spectra.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram illustrating an apparatus for predicting an absorbance spectrum according to an embodiment of the present invention;

FIGS. 2 and 3 are illustrative diagrams illustrating an operation of predicting absorbance spectra of first and second dyes according to an embodiment of the present invention;

FIGS. 4 and 5 are illustrative diagrams illustrating an operation of correcting first and second absorbance spectra according to an embodiment of the present invention;

FIG. 6 is an illustrative diagram illustrating a result of predicting a third absorbance spectrum according to an embodiment of the present invention;

FIGS. 7A-7E are the illustrative diagram illustrating an entire process of predicting an absorbance spectrum according to an embodiment of the present invention;

FIG. 8 is a flowchart showing a method for predicting an absorbance spectrum according to an embodiment of the present invention; and

FIG. 9 is a flowchart showing a dye prescription correction method for a blended fabric according to an embodiment of the present invention.

### DETAILED DESCRIPTION OF EXEMPLARY EMBODIMENTS

Hereinafter, embodiments of an apparatus and method for predicting an absorbance spectrum according to the present invention will be described with reference to the accompanying drawings. Thicknesses of lines and sizes of components illustrated in the drawings, for example, may be exaggerated for the sake of clarity and convenience of description. Further, terms to be described below are terms defined in consideration of functions in the present invention, and may vary depending on the intention or practice of users or operators. Therefore, definitions of these terms should be made based on the content throughout the present specification.

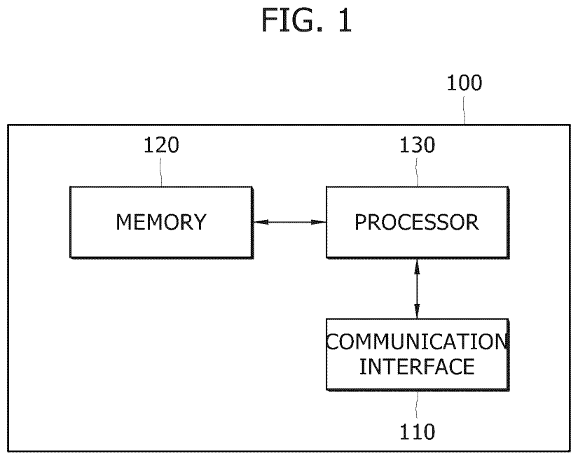

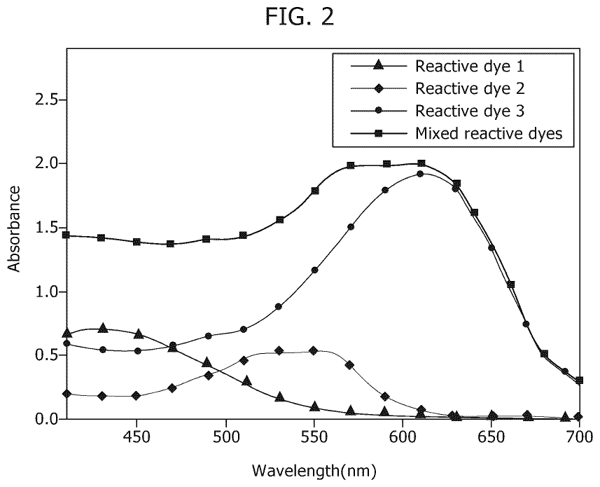

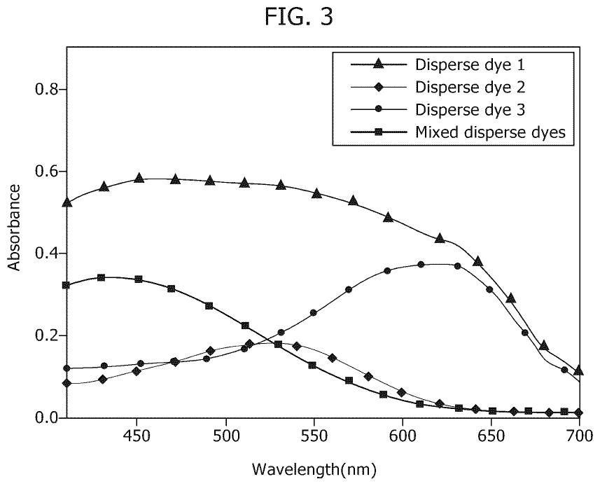

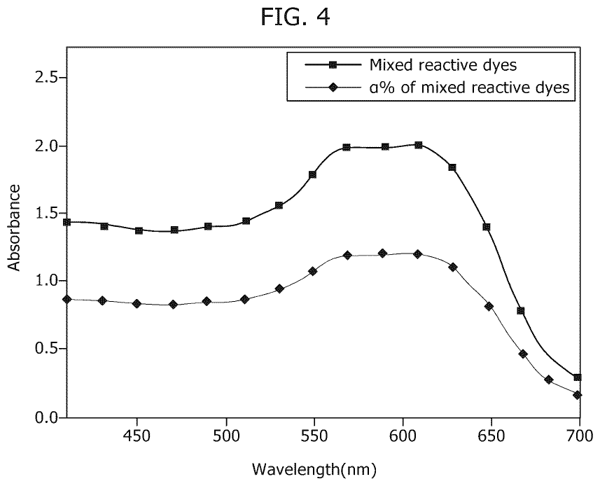

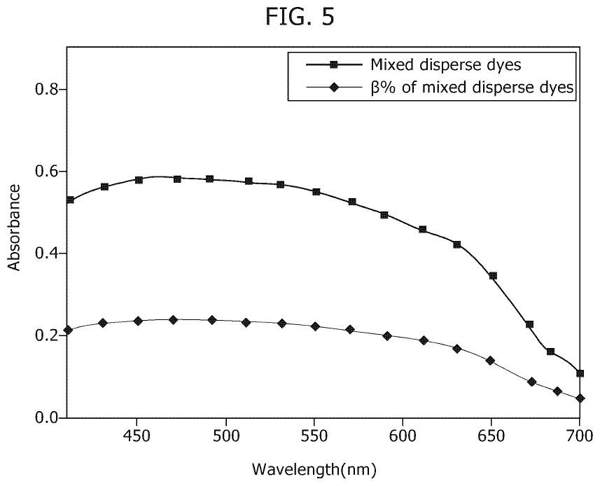

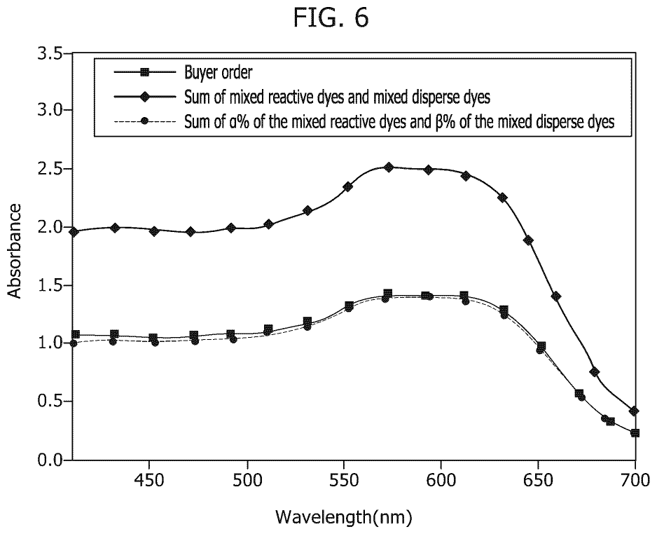

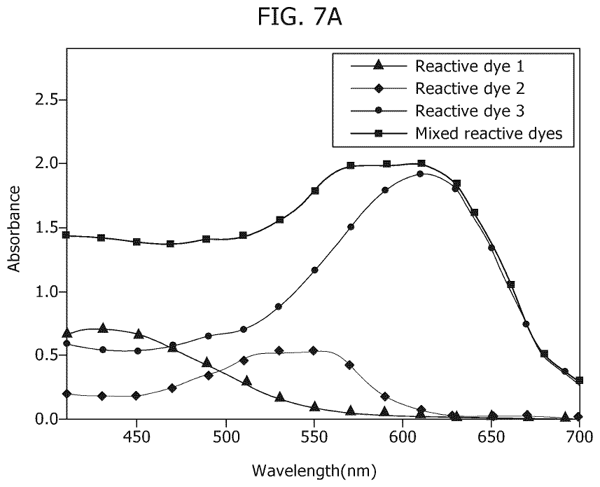

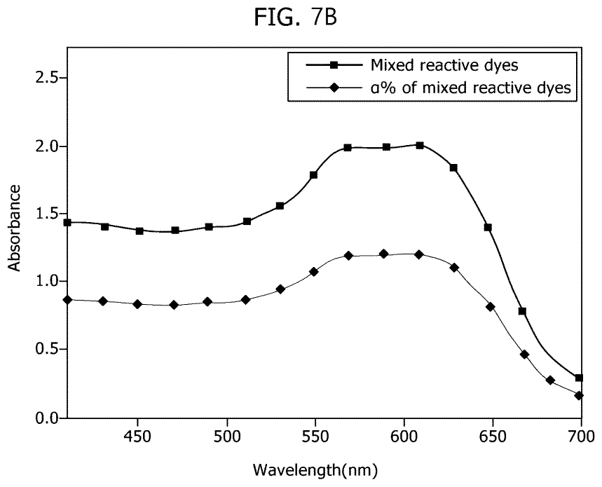

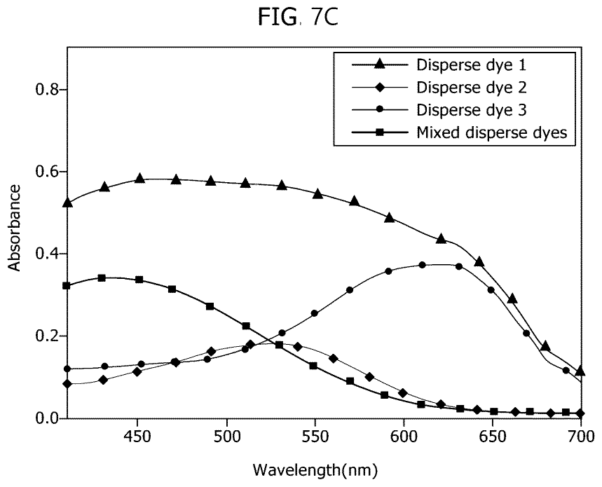

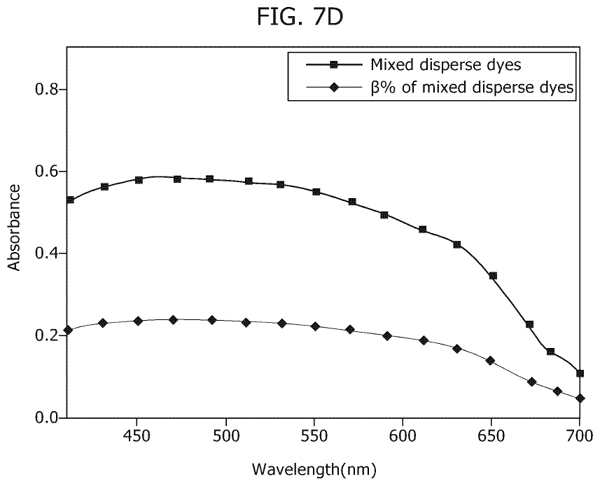

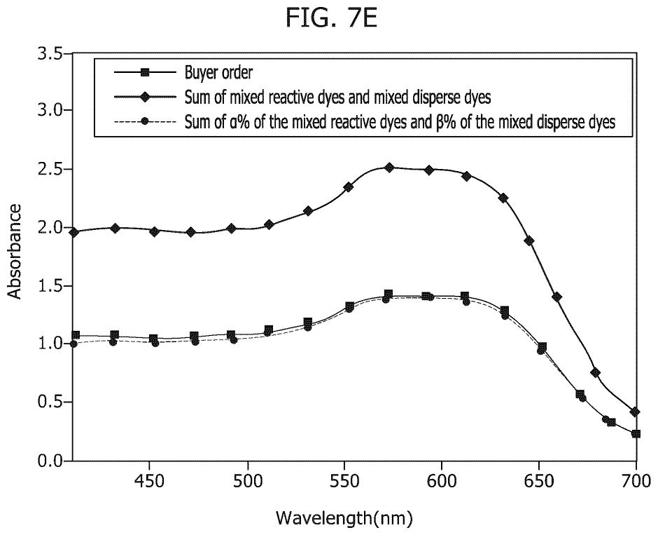

FIG. 1 is a block diagram illustrating an apparatus for predicting an absorbance spectrum according to an embodiment of the present invention, FIGS. 2 and 3 are illustrative diagrams illustrating an operation of predicting absorbance spectra of first and second dyes according to an embodiment of the present invention, FIGS. 4 and 5 are illustrative diagrams illustrating an operation of correcting first and second absorbance spectra according to an embodiment of the present invention, FIG. 6 is an illustrative diagram illustrating a result of predicting a third absorbance spectrum according to an embodiment of the present invention, and FIG. 7 is an illustrative diagram illustrating an entire process of predicting an absorbance spectrum according to an embodiment of the present invention.

Referring to FIG. 1, an apparatus for predicting an absorbance spectrum 100 according to an embodiment of the present invention may include a communication interface 110, a memory 120, and a processor 130. The apparatus for predicting an absorbance spectrum 100 according to the embodiment of the present invention may further include various components in addition to the components illustrated in FIG. 1, or may not include some of the above components.

The communication interface 110 may perform communication with an external device. The communication interface 110 may perform communication with various types of external devices according to various types of communication schemes.

The memory 120 may store at least one command that is executed by the processor 130. The memory 120 may be implemented as a volatile storage medium and/or a nonvolatile storage medium, such as a read-only memory (ROM) and/or a random access memory (RAM). The memory 120 may store various types of information that are required during the operation of the processor 130. The memory 120 may store various types of information that are calculated during the operation of the processor 130.

The processor 130 may be operatively connected to the communication interface 110 and the memory 120. The processor 130 may be implemented by a central processing unit (CPU) or a system on chip (SoC), and the processor 130 may control a plurality of hardware or software components connected to the processor 130 by driving an operating system or an application and may perform various data processing and calculations. The processor 130 may be configured to execute the at least one command stored in the memory 120 and store execution result data in the memory 120.

The processor 130 may predict an absorbance spectrum of a target blended fabric. The target blended fabric is a blended fabric of which the absorbance spectrum is to be predicted, and may include at least two types of fabrics (fibers). In the present specification, for the convenience of description, it is assumed that the target blended fabric includes two types of fabrics (a first fabric and a second fabric). The absorbance spectrum may be information in which absorbance according to a wavelength is recorded.

The processor 130 may predict a first absorbance spectrum, which is an absorbance spectrum for a first dye that is used for dyeing of a first fabric, predict a second absorbance spectrum, which is an absorbance spectrum for a second dye that is used for dyeing of a second fabric, correct the first and second absorbance spectra based on a blending ratio of the target blended fabric, and predict a third absorbance spectrum, which is an absorbance spectrum of the target blended fabric, based on the corrected first and second absorbance spectra.

The first dye used for dyeing of the first fabric and the second dye used for dyeing of the second fabric may be determined by a known dye prescription technology or by a user. In the present embodiment, the first dye may be a hydrophilic dye, and the second dye may be a hydrophobic dye. The first dye may be a reactive dye such as a cotton dye. The second dye may be a disperse dye such as a polyester dye. However, the types of the first and second fabrics are not limited to the above-described embodiments, and both the first and second dyes may be hydrophilic dyes, or both the first and second dyes may be hydrophobic dyes.

Fibers can generally be classified into natural fibers and synthetic fibers (chemical fibers). Natural fibers include cotton, wool, silk, and the like, and synthetic fibers include nylon, polyester, acrylic fiber, and the like. Dyes that can be used for dyeing can be classified depending on a type of fiber. Dyes include direct dyes, indigo dyes, reactive dyes, azo dyes, and disperse dyes, and can be classified according to their characteristics. The dyes may be roughly classified into hydrophilic (water-soluble) dyes and hydrophobic (insoluble) dyes.

In the present embodiment, the first dye may be a single dye or a mixed dye obtained by mixing two or more single dyes. In the present embodiment, the second dye may also be a single dye or a mixed dye obtained by mixing two or more single dyes.

The processor 130 may predict the absorbance spectrum of the first dye using a preset mathematical model. In the present embodiment, the mathematical model is a model for predicting an absorbance spectrum, and when a type and concentration of each of monochromatic dyes are input, the mathematical model may calculate and output an absorbance spectrum for a mixed dye generated through a combination of the monochromatic dyes corresponding to such input information. The mathematical model may be created by analyzing reflectance (reflectivity) data and absorbance data (spectrum) of each of the dyes through quantitative analysis and regression analysis, and may be created in advance and stored in the memory 120.

According to another embodiment, the processor 130 may predict the absorbance spectrum of the first dye from reflectance data (reflectivity data) for the first dye. Here, the reflectance data may be information in which reflectance according to a wavelength is recorded.

The processor 130 may receive the reflectance data for the first dye via the communication interface 110. The processor 130 may calculate the absorbance from the reflectance using Equation 1 below.

$$A = \frac{\text{?}}{log R} + α \qquad [ Equation 1 ]$$

$$\text{?} \text{indicates text missing or illegible when filed}$$

Here, A may be absorbance, R may be a percentage (that is, % R) for reflectivity, and a may be a constant.

When the first dye is a mixed dye, the processor 130 may predict the absorbance spectrum of the first dye from reflectance data for each of the monochromatic dyes that are used for production of the first dye. The processor 130 may predict the absorbance spectrum of the first dye by performing a process of predicting the absorbance spectrum from the reflectance data on each of the monochromatic dyes that are used for production of the first dye and summing absorbance spectra for the respective monochromatic dyes in consideration of a mixing ratio. The monochromatic dyes used for production of the first dye and the mixing ratio may be determined by a dye prescription, and the processor 130 may receive information on the monochromatic dyes used for production of the first dye and the mixing ratio via the communication interface 110.

For example, assuming that the monochromatic dyes used for production of the first dye are dye A (with a concentration of B %) and dye C (with a concentration of D %), and a mixing ratio of dye A (with a concentration of B %) and dye C (with a concentration of D %) is 2:1, the processor 130 may predict the absorbance spectrum of the first dye by repeatedly performing a process of summing a value of absorbance of dye A with a concentration of B % *2 and a value of absorbance of dye C with a concentration of D % *1 while changing a wavelength in a preset wavelength range (for example, visible light range).

For example, as illustrated in FIG. 2, assuming that the dyes used for production of the first dye are a first reactive dye (Reactive dye 1), a second reactive dye (Reactive dye 2), and a third reactive dye (Reactive dye 3), the processor 130 can derive the absorbance spectrum of the first dye (mixed reactive dye) from absorbance spectra for the first to third reactive dyes.

The processor 130 may predict the absorbance spectrum of the second dye using a preset mathematical model. According to another embodiment, the processor 130 may predict the absorbance spectrum of the second dye from reflectance data (reflectivity data) for the second dye. Here, the reflectance data for the second dye may be data obtained by recording reflectance (reflectivity) for the second dye for each wavelength. The processor 130 may receive the reflectance data for the second dye via the communication interface 110.

When the second dye is a mixed dye, the processor 130 may predict the absorbance spectrum of the second dye from reflectance data for each of monochromatic dyes used for production of the second dye. The processor 130 may predict the absorbance spectrum of the second dye by performing a process of predicting the absorbance spectrum from the reflectance data on each of the monochromatic dyes that are used for production of the second dye and summing absorbance spectra for the respective monochromatic dyes in consideration of a mixing ratio. The monochromatic dyes used for production of the second dye and the mixing ratio may be determined by a dye prescription, and the processor 130 may receive information on the monochromatic dyes used for production of the second dye and the mixing ratio via the communication interface 110.

For example, assuming that dyes used for production of the second dye are a first disperse dye (Disperse dye 1), a second disperse dye (Disperse dye 2), and a third disperse dye (Disperse dye 3), the processor 130 may derive the absorbance spectrum of the second dye (Mixed disperse dye) from absorbance spectra for the first to third disperse dyes, as illustrated in FIG. 3.

In various embodiments, the processor 130 may receive the absorbance spectra for the first and second dyes via the communication interface 110, instead of predicting the absorbance spectra for the first and second dyes.

The processor 130 may correct the first absorbance spectrum by performing a process of multiplying an absorbance value (absorbance at a specific wavelength) of the first fabric by a first percentage, which is a percentage of the first fabric in the target blended fabric, in an entire wavelength range (for example, the visible light range) of the first absorbance spectrum. For example, as illustrated in FIG. 4, the processor 130 may acquire a corrected absorbance spectrum (α % of mixed reactive dyes) of the first dye by multiplying the absorbance spectrum of the first dye (mixed reactive dye) by the first percentage (α %).

The processor 130 may correct the second absorbance spectrum by performing a process of multiplying an absorbance value of the second fabric by a second percentage, which is a percentage of the second fabric in the target blended fabric, in an entire wavelength range (for example, the visible light range) of the second absorbance spectrum. For example, as illustrated in FIG. 5, the processor 130 may acquire a corrected absorbance spectrum (β % of mixed reactive dyes) of the second dye by multiplying the absorbance spectrum of the second dye (mixed reactive dye) by the second percentage (β %).

The processor 130 may predict the third absorbance spectrum by performing a process of summing the corrected absorbance of the first dye and the corrected absorbance of the second dye in the entire wavelength range (for example, the visible light range) of the first and second absorbance spectra. In other words, the processor 130 may add the corrected first absorbance spectrum and the corrected second absorbance spectrum to calculate the third absorbance spectrum. When a dye prescription is appropriately made, the third absorbance spectrum (Sum of α % of mixed reactive dye and β % of mixed reactive dye) may be substantially similar to an absorbance spectrum (buyer order) for an electronic color value of a target color, as illustrated in FIG. 6.

The process of predicting the absorbance spectrum of the target blended fabric described above may be summarized as in FIG. 7. As illustrated in FIG. 7, the processor 130 may acquire a corrected first absorbance spectrum (FIG. 7B) by applying the first percentage to a first absorbance spectrum (FIG. 7A), acquire a corrected second absorbance spectrum (FIG. 7D) by applying the second percentage to a second absorbance spectrum (FIG. 7C), and acquire a third absorbance spectrum (FIG. 7E) from the corrected first and second absorbance spectra (FIGS. 7B and 7D). The absorbance spectrum of the blended fabric calculated through the above-described process may be used in a process of correcting a dye prescription for the blended fabric.

The processor 130 may compare the third absorbance spectrum with a fourth absorbance spectrum, which is the absorbance spectrum for the electronic color value of the target color (the color requested by the buyer), and correct the dye prescription for the target blended fabric based on a result of the comparison. The electronic color value of the target color may be a computer color matching (CCM) colorimetric value for the target color, which may represent a color determined according to a QTX file. The processor 130 may receive the fourth absorbance spectrum via the communication interface 110 or may receive reflectance data for the electronic color value of the target color via the communication interface 110 and predict the fourth absorbance spectrum from the received reflectance data. The dye prescription for the target blended fabric may be determined by a dye prescription system or a user, and may include information on a type and concentration of dyes used for dyeing of each of the fabrics constituting the target blended fabric, and a mixing ratio between the dyes. The processor 130 may receive information on the dye prescription for the target blended fabric via the communication interface 110.

The processor 130 can correct the dye prescription for the target blended fabric by performing at least one of an operation of replacing at least one of the first dye and the second dye with another dye, an operation of changing a concentration of at least one of the first dye and the second dye, and an operation of changing a mixing ratio of at least one of the first dye and the second dye.

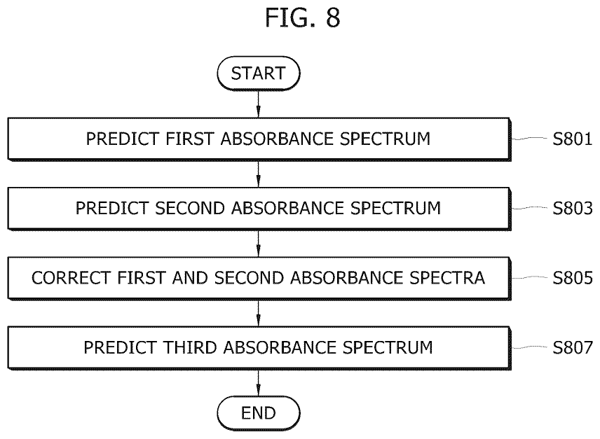

FIG. 8 is a flowchart showing a method for predicting an absorbance spectrum according to an embodiment of the present invention.

Hereinafter, a process of predicting an absorbance spectrum will be described with respect to the operation of the processor 130 with reference to FIG. 8. Some of the processes to be described below may be performed in a different order from that to be described below or may be omitted.

First, the processor 130 may predict the first absorbance spectrum, which is the absorbance spectrum of the first dye that is used for dyeing of the first fabric constituting the target blended fabric (S801). The first dye may be a hydrophilic dye. For example, the first dye may be a reactive dye such as a cotton dye. The processor 130 may predict the first absorbance spectrum from the reflectance data for each of the dyes (dyes determined through the dye prescription) used for production of the first dye.

Subsequently, the processor 130 may predict the second absorbance spectrum, which is the absorbance spectrum of the second dye used for dyeing of the second fabric constituting the target blended fabric (S803). The second dye may be a hydrophobic dye. For example, the second dye may be a disperse dye such as a polyester dye. The processor 130 may predict the second absorbance spectrum from the reflectance data for each of the dyes (dyes determined through the dye prescription) used for production of the second dye.

Subsequently, the processor 130 can correct the first and second absorbance spectra based on the blending ratio of the target blended fabric (S805). The processor 130 can correct the first absorbance spectrum by performing the process of multiplying the absorbance value of the first fabric by the first percentage, which is the percentage of the first fabric in the target blended fabric, in the entire wavelength range (for example, the visible light range) of the first absorbance spectrum. In other words, the processor 130 can correct the first absorbance spectrum by multiplying the first absorbance spectrum by the first percentage. The processor 130 may correct the second absorbance spectrum by performing the process of multiplying the absorbance value of the second fabric by the second percentage, which is the percentage of the second fabric in the target blended fabric, in the entire wavelength range (for example, the visible light range) of the second absorbance spectrum. In other words, the processor 130 can correct the second absorbance spectrum by multiplying the second absorbance spectrum by the second percentage.

In various embodiments, the processor 130 may additionally correct the first absorbance spectrum by multiplying the first absorbance spectrum by a preset third percentage or summing the first absorbance spectrum and the preset third percentage. Similarly, the processor 130 may additionally correct the first absorbance spectrum by multiplying the second absorbance spectrum by a preset fourth percentage or summing the second absorbance spectrum and the preset fourth percentage.

Subsequently, the processor 130 may predict the third absorbance spectrum, which is the absorbance spectrum of the target blended fabric, based on the corrected first and second absorbance spectra (S807). The processor 130 may predict the third absorbance spectrum by performing a process of summing the absorbance value of the first dye and the absorbance value of the second dye in the entire wavelength range (for example, the visible light range) of the first and second absorbance spectra. In other words, the processor 130 may predict the third absorbance spectrum by summing the corrected first absorbance spectrum and the corrected second absorbance spectrum.

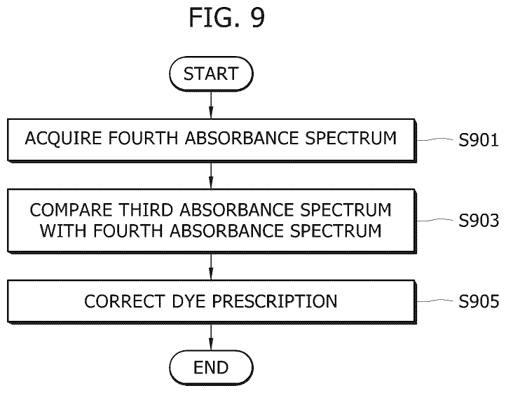

FIG. 9 is a flowchart showing a method for correcting the dye prescription for the blended fabric according to an embodiment of the present invention.

Hereinafter, a process of correcting the dye prescription for the blended fabric (or each of fabrics constituting the blended fabric) will be described with respect to operation of the processor 130 with reference to FIG. 9. Some of the processes to be described below may be performed in a different order from that to be described below or may be omitted. A process of FIG. 9 may be performed after the process of FIG. 8 is completed.

First, the processor 130 may acquire the fourth absorbance spectrum, which is the absorbance spectrum for the electronic color value of the target color (S901). The processor 130 may receive the fourth absorbance spectrum via the communication interface 110 or may receive reflectance data for the electronic color value of the target color via the communication interface 110 and predict the fourth absorbance spectrum from the received reflectance data.

Next, the processor 130 may compare the third absorbance spectrum (the absorbance spectrum of the blended fabric predicted through the process of FIG. 7) with the fourth absorbance spectrum (S903). In this case, the processor 130 may compare the third absorbance spectrum with the fourth absorbance spectrum with respect to a wavelength at which the absorbance is maximized, a maximum absorbance (an absorbance value at the wavelength at which the absorbance is maximized), or the like. The processor 130 may also confirm an accumulated value of an absorbance difference in the preset wavelength range (for example, the visible light range).

Subsequently, the processor 130 can correct the dye prescription for the target blended fabric based on a result of comparing the third absorbance spectrum with the fourth absorbance spectrum (S905). For example, when a difference between the third absorbance spectrum and the fourth absorbance spectrum is equal to or larger than a predetermined value in terms of the wavelength at which the absorbance is maximized or the maximum absorbance, the processor 130 may perform at least one of an operation of replacing at least one of the first dye and the second dye with another dye, an operation of changing a concentration of at least one of the first dye and the second dye, and an operation of changing a mixing ratio of at least one of the first dye and the second dye.

As described above, according to the present invention, it is possible to predict the absorbance spectrum of the blended fabric based on the absorbance spectrum of each of the fabrics constituting the blended fabric and the blending ratio of the blended fabric, and provide the absorbance spectrum to a user. Further, according to the present invention, it is possible to correct the dye prescription for the blended fabric based on a result of predicting the absorbance spectrum of the blended fabric.

Although the present invention has been described with reference to the embodiments illustrated in the drawings, this is merely an example, and it will be understood by those skilled in the art that various variations and other equivalent embodiments can be made from the example. Therefore. the technical protection scope of the present invention should be defined by the following claims.

What is claimed is:

1. An apparatus for predicting an absorbance spectrum of a blended fabric including a first fabric and a second fabric, the apparatus comprising:
a memory configured to store at least one command; and
a processor configured to perform the at least one command stored in the memory,
wherein the processor predicts a first absorbance spectrum, which is an absorbance spectrum for a first dye used for dyeing of the first fabric, predicts a second absorbance spectrum, which is an absorbance spectrum for a second dye used for dyeing of the second fabric, corrects the first and second absorbance spectra based on a blending ratio of the blended fabric, and predicts a third absorbance spectrum, which is an absorbance spectrum of the blended fabric, based on the corrected first and second absorbance spectra.

2. The apparatus for predicting an absorbance spectrum of claim 1,
wherein the first dye is a hydrophilic dye, and
the second dye is a hydrophobic dye.

3. The apparatus for predicting an absorbance spectrum of claim 1, wherein the first and second dyes are a single dye or a mixed dye.

4. The apparatus for predicting an absorbance spectrum of claim 1, wherein the processor predicts an absorbance spectrum of the first dye from information on a type and concentration of each of monochromatic dyes used for production of the first dye using a preset mathematical model.

5. The apparatus for predicting an absorbance spectrum of claim 1, wherein the processor corrects the first absorbance spectrum by performing a process of multiplying an absorbance value by a first percentage, which is a percentage of the first fabric in the blended fabric, in an entire wavelength range of the first absorbance spectrum, and corrects the second absorbance spectrum by performing a process of multiplying the absorbance value by a second percentage, which is a percentage of the second fabric in the blended fabric, in an entire wavelength range of the second absorbance spectrum.

6. The apparatus for predicting an absorbance spectrum of claim 1, wherein the processor predicts the third absorbance spectrum by performing a process of summing an absorbance value of the first dye and an absorbance value of the second dye in an entire wavelength range of the first and second absorbance spectra.

7. The apparatus for predicting an absorbance spectrum of claim 1, wherein the processor compares the third absorbance spectrum with a fourth absorbance spectrum, which is an absorbance spectrum for an electronic color value of a target color, and corrects a dye prescription for the blended fabric based on a result of the comparison.

8. The apparatus for predicting an absorbance spectrum of claim 7, wherein the processor corrects the dye prescription by performing at least one of an operation of replacing at least one of the first dye and the second dye with another dye, an operation of changing a concentration of at least one of the first dye and the second dye, and an operation of changing a mixing ratio of at least one of the first dye and the second dye.

9. A method for predicting an absorbance spectrum of a blended fabric including a first fabric and a second fabric, which is performed in a computing device including a processor, the method comprising:
predicting a first absorbance spectrum, which is an absorbance spectrum of a first dye used for dyeing of the first fabric;
predicting a second absorbance spectrum, which is an absorbance spectrum for a second dye used for dyeing of the second fabric;
correcting the first and second absorbance spectra based on a blending ratio of the blended fabric; and
predicting a third absorbance spectrum, which is the absorbance spectrum of the blended fabric, based on the corrected first and second absorbance spectra.

10. The method for predicting an absorbance spectrum of claim 9,
wherein the first dye is a hydrophilic dye, and
the second dye is a hydrophobic dye.

11. The method for predicting an absorbance spectrum of claim 9, wherein the first and second dyes are a single dye or a mixed dye.

12. The method for predicting an absorbance spectrum of claim 9, wherein the predicting of the first absorbance spectrum includes predicting an absorbance spectrum of the first dye from information on a type and concentration of each of monochromatic dyes used for production of the first dye using a preset mathematical model.

13. The method for predicting an absorbance spectrum of claim 9, wherein the correcting of the first and second absorbance spectra includes performing a process of multiplying an absorbance value by a first percentage, which is a percentage of the first fabric in the blended fabric, in an entire wavelength range of the first absorbance spectrum, and performing a process of multiplying the absorbance value by a second percentage, which is a percentage of the second fabric in the blended fabric, in an entire wavelength range of the second absorbance spectrum.

14. The method for predicting an absorbance spectrum of claim 9, wherein the predicting of the third absorbance spectrum includes performing a process of summing the absorbance of the first dye and the absorbance of the second dye in an entire wavelength range of the first and second absorbance spectra.

15. The method for predicting an absorbance spectrum of claim 9, further comprising:
comparing the third absorbance spectrum with a fourth absorbance spectrum, which is an absorbance spectrum for an electronic color value of a target color; and
correcting a dye prescription for the blended fabric based on a result of the comparison.

16. The method for predicting an absorbance spectrum of claim 15, wherein the correcting of the dye prescription includes performing at least one of an operation of replacing at least one of the first dye and the second dye with another dye, an operation of changing a concentration of at least one of the first dye and the second dye, and an operation of changing a mixing ratio of at least one of the first dye and the second dye.

17. An apparatus for predicting an absorbance spectrum of a blended fabric including a first fabric and a second fabric, the apparatus comprising:
a communication interface; and
a processor connected to the communication interface,
wherein the processor receives a first absorbance spectrum, which is an absorbance spectrum of a first dye used for dyeing of the first fabric and a second absorbance spectrum, which is an absorbance spectrum for a second dye used for dyeing of the second fabric via the communication interface, corrects the first and second absorbance spectra based on a blending ratio of the blended fabric, and predicts a third absorbance spectrum, which is an absorbance spectrum of the blended fabric, based on the corrected first and second absorbance spectra.

18. The apparatus for predicting an absorbance spectrum of claim 17,
wherein the first dye is a hydrophilic dye, and
the second dye is a hydrophobic dye.

19. The apparatus for predicting an absorbance spectrum of claim 17, wherein the processor corrects the first absorbance spectrum by multiplying the first absorbance spectrum by a first percentage, which is a percentage of the first fabric in the blended fabric, and corrects the second absorbance spectrum by multiplying the second absorbance spectrum by a second percentage, which is a percentage of the second fabric in the blended fabric.

20. The apparatus for predicting an absorbance spectrum of claim 17, wherein the processor predicts the third absorbance spectrum by summing the corrected first and second absorbance spectra.
